(19) United States
(12) Patent Application Publication
(10) Pub. No.: US 2022/0071812 A1
(43) Pub. Date: Mar. 10, 2022

(54) PATTERNING OF A STRANDED ELASTIC LAMINATE

(71) Applicant: Kimberly-Clark Worldwide, Inc., Neenah, WI (US)

(72) Inventors: Shawn E. Jenkins, Suwanee, GA (US); Howard M. Welch, Woodstock, GA (US); Susan E. Shawver, Buford, GA (US); James R. Fitts, Jr., Gainesville, GA (US); David J. Reader, Appleton, WI (US); Steven W. Fitting, Acworth, GA (US); Vikram S. Kaul, Atlanta, GA (US)

(21) Appl. No.: 17/415,991

(22) PCT Filed: Dec. 18, 2019
(86) PCT No.: PCT/US2019/067081

§ 371 Date: Jun. 18, 2021

Related U.S. Application Data
(60) Provisional application No. 62/783,193, filed on Dec. 20, 2018.

(51) Int. Cl. A61F 13/49; A61F 13/15

(52) U.S. Cl. CPC A61F 13/4902; A61F 13/15593; A61F 13/15699; A61F 13/15203; A61F 2013/15357; A61F 2013/49023; A61F 2013/49031; A61F 2013/15406; A61F 2013/49025

(57) ABSTRACT

The present disclosure provides for an apparatus for using a patterned stranded elastic laminate. The stranded elastic laminate is patterned by an anvil roll. The current disclosure also includes a process for making a patterned elastic laminate The patterned elastic laminate provides an aesthetic effect and is visible at least in both the stretched and unstretched states.

This application claims priority from U.S. provisional Patent Application Ser. No. 62/783193 filed on 20 Dec. 2018, the entire contents of which are incorporated herein by reference.

### TECHNICAL FIELD

The present disclosure provides for a more efficient and versatile patterned elastic laminate. The elastic laminate is patterned by an anvil roll. The current disclosure also includes processes for making a patterned elastic laminate.

### BACKGROUND OF THE DISCLOSURE

The current invention is specifically directed to an elastic strand laminate made up of elastic strands self-adhered to one or more facing sheets. Personal care garments often include elasticized portions to create a gasket-like fit around certain openings, such as waist openings and leg openings. Elastic laminates may be used in the manufacture of such garments to avoid complicated elastic-attachment steps during the garment manufacturing process.

One type of elastomeric laminate is a stretch-bonded laminate that includes elastic strands produced from an extruder and bonded to a facing sheet or sheets using a hot melt adhesive. Laminates including pre-made elastic strands can be processed online but require an elastic attachment adhesive with high add-on in order to reduce strand slippage. The cost of making stretch-bonded laminates can be relatively high due to the cost of the facing sheet or sheets, plus the cost of the elastic strands, plus the cost of the adhesive. Another type of elastomeric laminate may be made using a vertical filament laminate-stretch-bonded laminate (VFL-SBL) process. However, the VFL-SBL process must be in off-line operation due to process complexity.

Elastomeric adhesive compositions are multifunctional in the sense that they function as an elastomer in a nonwoven composite while also serving as a hot melt adhesive for bonding substrates. Elastomeric adhesive compositions in the form of elastomeric adhesive films are currently recognized as suitable for use in the manufacture of personal care articles. More particularly, elastomeric adhesive compositions may be used to bond facing materials, such as spunbond, to one another while simultaneously elasticizing the resulting laminate. The resulting laminate can be used to form an elastomeric portion of an absorbent article, such as a region surrounding a waist opening and/or a leg opening.

Non-woven elastic adhesive film laminates may require high output of adhesive add-on to achieve a tension target for product application. High add-on of the film laminate may generate a bulky, thick feel and appearance, and high cost. Furthermore, the high adhesive output requirement for the film formation would make on-line processing even more difficult due to the limitation of hot melt equipment output capacity. Also, such film lamination processes are relatively complex and need more precise control than strand lamination since a film edge thinning effect may cause the film to break during stretching.

Some elastomeric adhesive compositions lose their adhesiveness when the compositions are stretched and then bonded between two nonwoven substrates. The elasticity of these elastomeric adhesive compositions (in terms of tension decay) is negatively affected when laminates including the compositions are aged at elevated temperatures, for example around 54 degrees Celsius, which is commonly experienced under hot boxcar storage conditions. It appears that the poor tension and adhesion of such elastomeric adhesive compositions results from the chosen base polymer, tackifier, and plasticizer chemistries as well as the unbalanced ratio of polymer to low molecular weight species in the formulation.

Accordingly, there is a need or desire for an elastic laminate having improved elastic and adhesion properties which may be made in a relatively simple process with reduced raw material usage while still providing adequate tension for product application.

### SUMMARY OF THE DISCLOSURE

The current disclosure provides for a more efficient and versatile patterned elastic laminate wherein an elastic layer is placed between and attached to two facing layers that are made by meltblown, coform® or spunbond processes. The elastic layer includes one or more elastic strands. After the elastic layers are attached, the facing layers are gathered together when the elastic strands are in the relaxed state. The elastic laminate includes flattened areas that are visible when the laminate is in either the relaxed state or the stretched state. It is in the relaxed state that the elastic laminate is embossed.

The current disclosure also includes a process for making a patterned elastic laminate. The process includes stretching elastic strands, positioning the strands between two facing layers and attaching the stretched strands to the facing layers to form an elastic laminate. The elastic laminate is at least partially relaxing to come together in the facing layers. The relaxed elastic laminate is then passed through the nip of a pattern or anvil set of rolls to be embossed.

The current disclosure also discloses a process for making a patterned elastic laminate from stretching elastic strands, positioning the strands between two facing layers and attaching the stretched strands to the facing layers to form an elastic laminate. The process also includes keeping the elastic strands in the stretched state. The process further includes embossing the relaxed elastic laminate by passing it through the nip of a pattern or anvil set of rolls.

Another embodiment of the invention discloses an elastic nonwoven laminate having a machine direction (MD) and a cross-machine direction (CD). The elastic nonwoven laminate includes an unstretched or stretched plurality of strands of an elastomeric construction adhesive composition. The composition is self-adhered to at least a first nonwoven facing or a second nonwoven facing layer, wherein the first nonwoven facing layer comprises a first polyolefin and the first nonwoven facing is meltblown or spunbond. The first nonwoven facing layer and the second nonwoven facing layer are simultaneously grooved in the MD or CD to decouple the first nonwoven facing layer and the second nonwoven facing layer from the elastic strands. The grooves in the first nonwoven facing layer correspond with grooves in the second nonwoven facing layer and an outer surface of each of the first nonwoven facing layers and the second nonwoven facing layers are post-bonded after the first nonwoven facing layers and the second nonwoven facing layers are simultaneously grooved such that from about 1 groove per inch to about 20 grooves per inch are present on the outer surface of each of the first nonwoven facing layer and the second nonwoven facing layer after post-bonding. The outer surface of each of the first nonwoven facing layer and the second nonwoven facing layer are embossed in a pattern comprising embossed areas, wherein embossed areas of the first nonwoven facing layer and the second nonwoven facing layer are non-fibrous. The elastic nonwoven laminate has a percent elongation of at least about 200 percent in the CD, wherein the first nonwoven facing layer and the second nonwoven facing layer each exhibit improved elastic and adhesion properties.

In an additional embodiment of the present disclosure, a process for making a patterned elastic laminate is disclosed. The process includes stretching a plurality of elastic strands, positioning the strands between two or more nonwoven facing layers; attaching the stretched strands to the two or more nonwoven facing layers to form an elastic laminate thereby partially relaxing the elastic laminate; embossing the relaxed elastic laminate by passing it through the nip of an anvil set of rolls.

### BRIEF DESCRIPTION OF DRAWINGS

The disclosure may be more completely understood in consideration of the following detailed description of various embodiments in connection with the accompanying drawings, in which:

FIG. 1 shows a plane view of an elastic strand laminate.

FIG. 2 depicts a cross-sectional view of the elastic strand laminate of FIG. 1.

FIG. 3 shows the material disclosed therein has multifilament elastic strands. When the material is stretched the sample loses little to no elasticity.

FIG. 4 depicts the material disclosed therein has monofilament elastic strands. When the material is stretched the sample loses its elasticity.

### DETAILED DESCRIPTION OF THE DISCLOSURE

When introducing elements of the present disclosure or the preferred embodiment(s) thereof, the articles “a”, “an”, and “the” are intended to mean that there are one or more of the elements.

As used herein, the term “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

As used herein, the terms “machine direction” or “MD” generally refers to the direction in which a material is produced. The term “cross-machine direction” or “CD” refers to the direction perpendicular to the machine direction.

As used herein, the term “nonwoven” generally refers to a web having a structure of individual fibers or threads which are interlaid, but not in an identifiable manner as in a knitted fabric. Examples of suitable nonwoven fabrics or webs include, but are not limited to, meltblown webs, spunbond webs, bonded carded webs, airlaid webs, coform webs, hydraulically entangled webs, and so forth.

As used herein, the term “meltblown” generally refers to a nonwoven web that is formed by a process in which a molten thermoplastic material is extruded through a plurality of fine, usually circular, die capillaries as molten fibers into converging high velocity gas (e.g. air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin, et al., which is incorporated herein in its entirety by reference thereto for all purposes. Generally speaking, meltblown fibers may be microfibers that are substantially continuous or discontinuous, generally smaller than 10 microns in diameter, and generally tacky when deposited onto a collecting surface.

As used herein, the term “spunbond” generally refers to a web containing small diameter substantially continuous fibers. The fibers are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spunbond webs is described and illustrated, for example, in U.S. Pat. No. 3,338,992 to Kinney, U.S. Pat. No. 3,341,394 to Kinney, U.S. Pat. No. 3,502,538 to Levy, U.S. Pat. No. 3,502,763 to Hartman, U.S. Pat. No. 3,542,615 to Dobo, et al., U.S. Pat. No. 3,692,618 to Dorschner, et al., U.S. Pat. No. 3,802,817 to Matsuki, et al., U.S. Pat. No. 4,340,563 to Appel, et al., and U.S. Pat. No. 5,382,400 to Pike et al., which are incorporated herein in their entirety by reference thereto for all purposes. Spunbond fibers are generally not tacky when they are deposited onto a collecting surface. As such, the fibers may be bonded together after deposition onto a collecting surface in order to integrate the fibers. Spunbond fibers may sometimes have diameters less than about 40 microns, and are often between about 5 to about 20 microns.

As used herein, the term “elastic tension” refers to the amount of force per unit width required to stretch an elastic material (or a selected zone thereof) to a given percent elongation.

As used herein, the term “plurality” refers to one or more strands.

As used herein, the term “elastomeric” and “elastic” and refers to a material that, upon application of a stretching force, is stretchable in at least one direction (such as the CD direction), and which upon release of the stretching force, contracts/returns to approximately its original dimension. For example, a stretched material may have a stretched length that is at least 50 percent greater than its relaxed unstretched length, and which will recover to within at least 50 percent of its stretched length upon release of the stretching force. A hypothetical example would be a one (1) inch sample of a material that is stretchable to at least 1.50 inches and which, upon release of the stretching force, will recover to a length of not more than 1.25 inches. Desirably, the material contracts or recovers at least 50 percent, and even more desirably, at least 80 percent of the stretched length.

As used herein, the term “elongation” refers to the capability of an elastic material to be stretched a certain distance, such that greater elongation refers to an elastic material capable of being stretched a greater distance than an elastic material having lower elongation.

As used herein, the term “film” refers to a thermoplastic film made using a film extrusion process, such as a cast film or blown film extrusion process. The term includes apertured films, slit films, and other porous films which constitute liquid transfer films, as well as films which do not transfer liquid.

As used herein, the term “absorbent article” includes diapers, training pants, swim wear, absorbent underpants, adult incontinence products, feminine hygiene products, and the like.

As used herein, the term “layer” when used in the singular may have the dual meaning of a single element or a plurality of elements.

As used herein, the term “polymers” include, but are not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the material. These configurations include, but are not limited to isotactic, syndiotactic and atactic symmetries.

As used herein, the term “vertical filament stretch-bonded laminate” or “VF SBL” refers to a stretch-bonded laminate made using a continuous vertical filament process, as described herein.

The present invention is directed to an elastic strand laminate having superior elastic and adhesion properties, and a method of making such laminates. The laminate can be incorporated into any suitable article, such as an absorbent article. More particularly, the elastic strand laminate is suitable for use in diapers, training pants, swim wear, absorbent underpants, adult incontinence products, feminine hygiene products, protective medical gowns, surgical medical gowns, caps, gloves, drapes, face masks, laboratory coats, and coveralls.

A number of elastomeric components are known for use in the design and manufacture of such articles. For example, disposable absorbent articles are known to contain elasticized leg cuffs, elasticized waist portions, and elasticized fastening tabs. The elastic strand laminate of this invention may be applied to any suitable article to form such elasticized areas.

The strands of the elastomeric adhesive composition can be self-adhered to one or more facing sheets, suitably between two facing sheets of spunbond, meltblown, film, or other facing material. The strands can be spaced apart on the facing sheet(s) by about 2 to about 18 strands per inch, with each of the strands having a diameter or width of between about 0.1 and about 0.25 inch. The elastic strand laminates of the invention suitably have a basis weight between about 20 and about 120 grams per square meter. The elastic strand laminates of the invention significantly improve the rate and extent of tension decay, as well as adhesion properties of the spunbond laminates compared to spunbond laminates including conventional elastomeric adhesive compositions. Furthermore, the elastic strand laminates of the invention require a lower output of adhesive add-on, compared to elastic film laminates, to achieve a tension target for product application which also results in less bulk and lower cost.

More specifically, the present invention is directed to a patterned stranded elastic nonwoven laminate wherein an elastic layer is placed between and attached to two facing layers that are made by meltblown, coform® or spunbond processes. The elastic layer includes one or more elastic strands. After the elastic layers are attached, the facing layers are gathered (come together) when the elastic strands are in the relaxed state this forming an elastic laminate. The elastic laminate includes flattened areas that are visible when the laminate is in either the relaxed state or the stretched state. The two facing layers may be nonwoven and may include a conventional polyolefin which may, in some embodiments, be combined with a polyolefin-based plastomer. Further, the nonwoven facing layers may be spunbond or meltblown. The laminate may be activated by grooving and then can be post-bonded. By activation, it is meant that the laminate's elasticity, attributed to the elastic film, is unlocked, such as by breaking portions of the nonwoven facing layer. It has been found that by using two or more elastic layers which include elastic strands as opposed to elastic film a desired level of compaction to prevent the fiber pull-out typically seen in groove-activated spunbond or meltblown webs can be achieved without sacrificing the elasticity, softness, loftiness, hand feel, and/or aesthetic appeal of the resulting laminate. Thus, a spunbond or meltblown elastic nonwoven laminate can be produced that can be reusable, such as in fastening/unfastening applications, due to the reduced occurrence of fiber pull-out, which also minimizes the “fuzziness” of the laminate despite utilizing grooving to activate the laminates instead of other activation methods, such as heat activation. As such, the elastic nonwoven laminate of the present invention can be used instead of bonded carded web-based elastic laminates. In this regard, various embodiments of the present invention will now be described in more detail.

The core layer of the elastic strand of the elastic nonwoven laminate of the present disclosure may provide the laminate with the desired elasticity. Any of a variety of thermoplastic elastomeric or plastomeric polymers may generally be employed in the two layers of the elastic strand of the elastic nonwoven laminate of the present disclosure. Such polymers include elastomeric polyesters, elastomeric polyurethanes, elastomeric polyamides, elastomeric copolymers, elastomeric polyolefins, and so forth. In one embodiment, for instance, a substantially amorphous block copolymer may be employed that contains blocks of a monoalkenyl arene and a saturated conjugated diene. Such block copolymers are particularly useful in the present invention due to their high degree of elasticity.

The elastic nonwoven laminate of the present disclosure may also include one or more nonwoven facing layers that may serve as an exterior surface of the laminate. The nonwoven facing layers, for instance, may comprise a nonwoven material, such as a spunbond web or a meltblown web. The spunbond or meltblown nonwoven facing can include a polyolefin, and, in some embodiments, can include a combination of a polyolefin and a polyolefin-based plastomer. For example, in some embodiments, the spunbond or meltblown nonwoven facing can include a polyethylene and a polyethylene-based plastomer or a polypropylene and a polypropylene-based plastomer. In other embodiments, the spunbond or meltblown nonwoven facing can include a combination of any of the following: polyethylene, polypropylene, a polyethylene-based plastomer, and/or a polypropylene-based plastomer.

Polyethylenes that can be used to form the nonwoven facing layer include conventional polyethylene and low density polyethylene (LDPE). Other suitable ethylene polymers are available from The Dow Chemical Company under the designations ASPUN™ (LLDPE), DOWLEX™ (LLDPE) and ATTANET™ (ULDPE). Other suitable ethylene polymers are described in U.S. Pat. No. 4,937,299 to Ewen, et al., U.S. Pat. No. 5,218,071 to Tsutsui et al., U.S. Pat. No. 5,272,236 to Lai, et al., and U.S. Pat. No. 5,278,272 to Lai, et al., which are incorporated herein in their entirety by reference thereto for all purposes.

In addition, polyethylene-based plastomers can be used in conjunction with the aforementioned polyethylenes when forming the spunbond or meltblown nonwoven facing layer. Such ethylene-based plastomers include ethylene-based copolymer plastomers available under the designation EXACT™ from ExxonMobil Chemical Company of Houston, Tex. Other suitable polyethylene plastomers are available under the designation ENGAGE™ and AFFINITY™ from Dow Chemical Company of Midland, Mich. An additional suitable polyethylene-based plastomer is an olefin block copolymer available from Dow Chemical Company of Midland, Mich. under the trade designation INFUSE™.

Of course, the present disclosure is by no means limited to the use of ethylene polymers. For instance, conventional polypropylene can be a component of the spunbond or meltblown nonwoven facing layer. Further, propylene plastomers may also be suitable for use in the nonwoven facing layers in combination with conventional polypropylene. Suitable plastomeric propylene polymers may include, for instance, copolymers or terpolymers of propylene, copolymers of propylene with an a-olefin (e.g., C₃-C₂₀), such as ethylene, 1-butene, 2-butene, the various pentene isomers, 1-hexene, 1-octene, 1-nonene, 1-decene, 1-unidecene, 1-dodecene, 4-methyl-1-pentene, 4-methyl-1-hexene, 5-methyl-1-hexene, vinylcyclohexene, styrene, etc. The comonomer content of the propylene polymer may be about 35 weight percent or less, in some embodiments from about 1 weight percent to about 20 weight percent, and in some embodiments, from about 2 weight percent to about 10 weight percent. Preferably, the density of the polypropylene (e.g., propylene/α-olefin copolymer) may be 0.91 g/cm³ or less, in some embodiments, from 0.85 g/cm³ to 0.88 g/cm³, and in some embodiments, from 0.85 g/cm³ to 0.87 g/cm³. Suitable propylene polymers are commercially available under the designations VISTAMAXX™ (e.g., 6102), a propylene-based elastomer from ExxonMobil Chemical Co. of Houston, Tex.; FINA™ (e.g., 8573) from Atofina Chemicals of Feluy, Belgium; TAFMER™ available from Mitsui Petrochemical Industries; and VERSIFY™ available from Dow Chemical Co. of Midland, Mich. Other examples of suitable propylene polymers are described in U.S. Pat. No. 5,539,056 to Yang, et al., U.S. Pat. No. 5,596,052 to Resconi, et al., and U.S. Pat. No. 6,500,563 to Datta, et al., which are incorporated herein in their entirety by reference thereto for all purposes.

Regardless of the particular combination of polyolefins and/or polyolefin-based plastomers employed in the nonwoven facing layer(s) of the present disclosure, a polyolefin can be present in the nonwoven facing layer(s) in an amount up to about 100 percent, such as an amount ranging from about 40 weight percent to about 100 weight percent, such as an mount ranging from about 50 weight percent to about 99 weight percent, such as an amount ranging from about 60 weight percent to about 98 weight percent based on the total weight of the nonwoven facing layer(s). Meanwhile, a polyolefin-based plastomer can be present in the nonwoven facing layer(s) in an amount ranging from about 0.5 weight percent to about 60 weight percent, such as from about 1 weight percent to about 50 weight percent, such as from about 2 weight percent to about 40 weight percent based on total weight of the nonwoven facing layers.

If desired, the nonwoven facing layer used to form the elastic nonwoven laminate of the present invention may have a multi-layer structure. Suitable multi-layered materials may include, for instance, spunbond/meltblown/spunbond (SMS) laminates and spunbond/meltblown (SM) laminates. Various examples of suitable SMS laminates are described in U.S. Pat. No. 4,041,203 to Brock et al., U.S. Pat. No. 4,374,888 to Bornslaeger, U.S. Pat. No. 4,766,029 to Brock et al., U.S. Pat. No. 5,169,706 to Collier et al., U.S. Pat. No. 5,213,881 to Timmons et al., and U.S. Pat. No. 5,464,688 to Timmons, et al., which are incorporated herein in their entirety by reference thereto for all purposes. In addition, commercially available SMS laminates may be obtained from Kimberly-Clark Corporation under the designations Spunguard(R) and Evolution(R).

Another example of a multi-layered structure is a spunbond web produced on a multiple spin bank machine in which a spin bank deposits fibers over a layer of fibers deposited from a previous spin bank. Such an individual spunbond nonwoven facing may also be thought of as a multi-layered structure. In this situation, the various layers of deposited fibers in the nonwoven web may be the same, or they may be different in basis weight and/or in terms of the composition, type, size, level of crimp, and/or shape of the fibers produced. As another example, a single nonwoven facing may be provided as two or more individually produced layers of a spunbond web, a meltblown web, etc., which have been bonded together to form the nonwoven facing. These individually produced layers may differ in terms of production method, basis weight, composition, etc. as discussed above.

The basis weight of each of the nonwoven facing layers may generally vary, such as from about 1 gsm to about 120 gsm, such as from about 5 gsm to about 80 gsm, such as from about 10 gsm to about 60 gsm, such as from about 15 gsm to about 40 gsm. When multiple nonwoven facing layers are utilized, such materials may have the same or different basis weights.

Any of a variety of techniques may be employed to laminate the nonwoven facing layers discussed above together to form the elastic nonwoven laminate of the present invention, including adhesive bonding, thermal bonding, ultrasonic bonding, microwave bonding, extrusion coating, and so forth. In one particular embodiment, nip rolls apply a pressure to the elastic film and nonwoven facing(s) to thermally bond the layers together. The rolls may be smooth and/or contain a plurality of raised bonding elements. In one embodiment, a laminate containing an elastic strand sandwiched between two nonwoven facing layers can be formed. The rolls used to join the strand to the nonwoven facing layers can be smooth chill rolls, and the nonwoven facing layers can be laminated to the strand by extrusion casting the elastic strand between two facing materials as the strand and facing materials pass through the nip between the chilled rolls. In another embodiment, an already-cast film can be disposed between the nonwoven facing layers and adhesively bonded to the nonwoven facing layers. Adhesives that can be employed can include BOSTIK™ H2494 available from Bostik Findley, Inc, of Wauwatosa, Wis. and REXTAC™ 2730 and 2723 available from Huntsman Polymers of Houston, Tex. The type and basis weight of the adhesive used will be determined on the elastic attributes desired in the final composite and end use. For instance, the basis weight of the adhesive may be from about 0.5 gsm to about 3 gsm, such as from about 0.75 gsm to about 1.75 gsm, such as from about 1 gsm to about 1.5 gsm. The adhesive may be applied to the nonwoven web facings and/or the elastic material prior to lamination using any known technique, such as by ribbon, slot, melt spray, of dot pattern adhesive systems.

Optionally, heat may be applied to the composite or laminate just prior to or during the application of the grooves to cause it to relax somewhat and ease extension. Heat may be applied by any suitable method known in the art, such as heated air, infrared heaters, heated nipped rolls, or partial wrapping of the laminate around one or more heated rolls or steam canisters, etc. Heat may also be applied to the grooved rolls themselves. It should also be understood that other grooved roll arrangement are equally suitable, such as two grooved rolls positioned immediately adjacent to one another. In another embodiment, the process may include a grooved roll that contacts a flat anvil roll which may have a deformable surface.

After the laminate has been formed via attaching the elastic nonwoven as discussed above, and after the nonwoven facing has been decoupled from the elastic strand via grooving to activate machine direction and or cross-machine direction stretchability of the laminate, typically, with spunbond or meltblown nonwoven facings, the fibers in the nonwoven facing can separate from each other, pull out, and create a “fuzzy” appearance. These facings can also have insufficient shear and peel properties for use in certain absorbent article applications, which can prevent the use of meltblown or spunbond nonwoven facings in laminates where fiber pullout is a concern, such as in materials utilizing reusable fastening/attachment mechanisms.

Meanwhile, post-bonding of an outer surface of the nonwoven facing material can reduce fiber pull out and the fuzzy appearance of meltblown and spunbond nonwoven facings in laminates that have been groove-activated so that such laminates can be used in applications with minimal fiber pullout, yet without sacrificing the softness or feel of the laminates, nor their elastic stretchability and recoverability. Post-bonding of an outer-facing surface of nonwoven facing layer can generally be accomplished in the present invention via a smooth calendar roll or via a patterned bonding technique (e.g., thermal point bonding, ultrasonic bonding, etc.) in which the laminate is supplied to a nip defined by at least one patterned roll. Thermal point bonding, for instance, typically employs a nip formed between two rolls, at least one of which is patterned. Ultrasonic bonding, on the other hand, typically employs a nip formed between a sonic horn and a patterned roll. Regardless of the technique chosen, the patterned roll can contain a plurality of bonding elements to bond the film to the nonwoven web material(s) and, in some embodiments, form apertures in the nonwoven facing, such as when the laminate is used as a side panel in an absorbent article and should be breathable. The size of the bonding elements may be specifically tailored to enhance bonding of the nonwoven facing and can also be selected to facilitate the formation of apertures in the nonwoven facing and, in some embodiments, the film layer of the laminate. For example, the bonding elements are typically selected to have a relatively large length dimension. The length dimension of the bonding elements may be from about 300 to about 5000 micrometers, in some embodiments from about 500 to about 4000 micrometers, and in some embodiments, from about 1000 to about 2000 micrometers. The width dimension of the bonding elements may likewise range from about 20 to about 500 micrometers, in some embodiments from about 40 to about 200 micrometers, and in some embodiments, from about 50 to about 150 micrometers. In addition, the “element aspect ratio” (the ratio of the length of an element to its width) may range from about 2 to about 100, in some embodiments from about 4 to about 50, and in some embodiments, from about 5 to about 20.

The size, shape, number, and configuration of openings in a patterned roll can be varied to meet the particular end-use needs of the pattern-unbonded nonwoven facing of the laminate being formed thereby. In order to reduce the incidence of fiber pull-out in the resulting laminate material, the size of openings in patterned roll may be dimensioned to reduce the likelihood that the entire length of the filaments or fibers forming an unbonded area will lie within a single unbonded area, Stated differently, fiber length should be selected to reduce the likelihood that the entire length of a given fiber or filament will fall within a single unbonded area. On the other hand, the desirability of restricting the size of the openings in patterned roll, and the unbonded areas formed thereby in the pattern-unbonded nonwoven facing, is counter-balanced by the need for the unbonded areas to have sufficient size to provide the required engagement areas for the hook elements of a complementary hook material, in applications where, for example, the elastic nonwoven laminates is used as part of a fastening system in an absorbent article. The bonding areas can also be minimized so that the resulting laminate material maintains a desired level of loftiness.

Circular openings have an average diameter ranging from about 0.050 inch (about 0.127 cm) to about 0.250 inch (about 0.635 cm), such as from about 0.130 inch (0.330 cm) to about 0.160 inch (0.406 cm), and a depth measured from the outermost surface of patterned roll of at least about 0.020 inch (about 0.051 cm), such as from about 0.060 inch (0.152 cm), are considered suitable in forming the pattern-unbonded nonwoven material of the present invention. While openings in patterned roll are circular, other shapes, such as ovals, squares, diamonds and the like can be advantageously employed.

The number or density of openings in patterned roll also can be selected to provide the requisite amount of engagement areas for, for instance, hook elements in an absorbent article, without unduly limiting the size of the continuous bonded areas and giving rise to increased incidence of fiber pull-out. Pattern rolls having an opening density in the range of from about 1 opening per square centimeter (cm²) to about 25 openings/cm², such as from about 5 openings/cm² to about 7 openings/cm², may be utilized to advantage in forming the pattern-unbonded nonwoven facing in the laminate of the present invention.

Moreover, the spacing between individual openings may be selected to enhance the hook engagement functionality of the resulting laminate including the pattern-unbonded nonwoven facing, which can, in some embodiments, be used as a loop material, without overly reducing the portion of the pattern-unbonded loop material occupied by continuous bonded areas, which serve to lessen fiber pull-out. Suitable inter-opening spacings for the embodiment shown can range from about 0.13 inch (about 3.30 mm) to about 0.22 inch (about 5.59 mm), centerline-to-centerline, in the machine and cross-machine directions,

The particular arrangement or configuration of openings in patterned roll is not considered critical, so long as in combination with the opening size, shape and density, the desired levels of surface integrity, loftiness, durability, peel strength, etc. can be achieved. For example, the individual openings are arranged in staggered rows. Other different configurations are considered within the scope of the present invention.

The portion of the outermost surface of the patterned roll occupied by continuous land areas likewise can be modified to satisfy the contemplated end-use application of the pattern-unbonded material. The degree of bonding imparted to the pattern-unbonded nonwoven facing of the laminate by the continuous land areas may be expressed as a percent bond area, which refers to the portion of the total plan area of at least one outer surface of a pattern-unbonded nonwoven facing that is occupied by bonded areas and unbonded areas. Stated generally, the lower limit on the percent bond area suitable for forming the pattern-unbonded nonwoven facing of the present invention is the point at which fiber pull-out excessively reduces the surface integrity and durability of the pattern-unbonded material. The required percent bond area will be affected by a number of factors, including the type(s) of polymeric materials used in forming the fibers or filaments of the nonwoven facing, whether the nonwoven facing is a single- or multi-layer fibrous structure, whether the nonwoven facing is unbonded or pre-bonded prior to passing into the pattern-unbonding assembly, and the like. Pattern-unbonded nonwoven facings having percent bond areas ranging from about 10 percent to about 60 percent, such as from about 15 percent to about 55 percent, such as from about 20 percent to about 50 percent based on the total surface area of the nonwoven facing, have been found suitable.

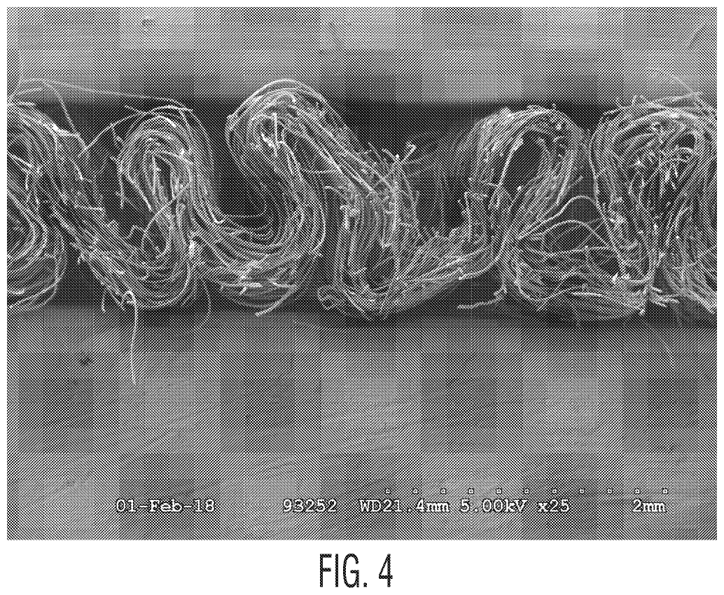

The temperature of the outer surface of patterned roll can be varied by heating or cooling relative to anvil roll. Heating and/or cooling may affect the features of the laminate(s) being processed and the degree of bonding of single or multiple laminates being passed through the nip formed between the counterrotating patterned roll and anvil roll. In the embodiment shown in FIG. 4, for example, both patterned roll and anvil roll are heated, desirably to the same bonding temperature. The specific ranges of temperatures to be employed in forming the pattern-unbonded nonwoven facing is dependent upon a number of factors, including the types of polymeric materials employed in forming the pattern-unbonded nonwoven facing, the inlet or line speed(s) of the nonwoven web(s) passing through the nip formed between patterned roll and anvil roll, and the nip pressure between patterned roll and anvil roll.

An anvil roll has an outer surface that is much smoother than a patterned roll, and preferably is smooth or flat. It is possible, however, for anvil roll to have a slight pattern on its outer surface and still be considered smooth or flat for purposes of the present invention. For example, if an anvil roll is made from or has a softer surface, such as resin impregnated cotton or rubber, it will develop surface irregularities, yet it will still be considered smooth or flat for purposes of the present invention. Such surfaces are collectively referred to herein as “flat”. An anvil roll provides the base for a patterned roll and the web or webs of material to contact. Typically, an anvil roll will be made from steel, or materials such as hardened rubber, resin-treated cotton or polyurethane.

Alternatively, an anvil roll may be replaced with a pattern roll having a pattern of continuous land areas defining a plurality of discrete, apertures or openings therein, as in the above-described patterned roll. In such case, the pattern-unbonding assembly would include a pair of counter-rotating pattern rolls which would impart a pattern of continuous bonded areas defining a plurality of discrete unbonded areas on both the upper and lower surfaces of the pattern-unbonded nonwoven loop material. Rotation of the opposedly positioned pattern rolls can be synchronized, such that the resulting unbonded areas on the surfaces of the pattern-unbonded material are vertically aligned or juxtaposed.

The pattern of the bonding elements is generally selected so that the nonwoven facing has a total bond area of less than about 50 percent (as determined by conventional optical microscopic methods), and in some embodiments, less than about 30 percent. The bond density is also typically greater than about 50 bonds per square inch, and in some embodiments, from about 75 to about 500 pin bonds per square inch. One suitable bonding pattern for use in the present invention is known as an “S-weave” pattern and is described in U.S. Pat. No. 5,964,742 to McCormack et al., which is incorporated herein in its entirety by reference thereto for all purposes. S-weave or wire-weave patterns typically have a bonding element density of from about 50 to about 500 bonding elements per square inch, and in some embodiments, from about 75 to about 150 bonding elements per square inch. Other bond patterns that may be used in the present invention are described in U.S. Pat. No. 3,855,046 to Hansen et al., U.S. Pat. No. 5,962,112 to Haynes et al., U.S. Pat. No. 6,093,665 to Sayovitz et al., U.S. Pat. No. 0,375,844 to Edwards, et al., D390,708 to Brown, and D428,267 to Romano et al., which are incorporated herein in their entirety by reference thereto for all purposes. Although the patterned rolls discussed above are generally utilized to bond the nonwoven facings of the present invention, such rolls, as briefly mentioned above, can also be used to form apertures in the nonwoven facings. In some embodiments, vacuum aperturing processes can also be used.

The selection of an appropriate bonding temperature (e.g., the temperature of a heated roll) will help melt and/soften the polymer(s) of the nonwoven facing at regions adjacent to the bonding elements. The softened polymer(s) may then flow and become displaced during bonding, such as by pressure exerted by the bonding elements. The displaced portions of the nonwoven facing can also fuse to other portions of the nonwoven facing, thereby reducing the fuzziness and reducing the fiber-pullout from the nonwoven facing typically experienced when bonded carded webs and other meltblown and spunbond nonwoven webs are utilized in a nonwoven facing. To achieve such bond formation on the nonwoven facing, the bonding temperature, pressure, and nip speed may be selectively controlled. For example, one or more rolls may be adjusted to a surface temperature of from about 18 degrees Celsius to about 149 degrees Celsius, in some embodiments from about 79 degrees Celsius to about 121 degrees Celsius, and in some embodiments, from about 82 degrees Celsius to about 116 degrees Celsius. Likewise, the pressure exerted by the bond rolls (“nip pressure”) during thermal bonding of the nonwoven facing may range from about 5 pound per square inch (psi) to about 100 psi, such as from about 10 psi to about 65 psi, such as from about 15 psi to about 60 psi, such as from about 20 psi to about 50 psi.

Further, in some embodiments, the post-bond temperature may range from about 87 degrees Celsius to about 98 degrees Celsius and, the post-bond pressure can range from about 10 psi to about 35 psi. In still other embodiments, the post-bonding can be carried out at ambient temperature, such as from about 65 degrees Cesius to about 23 degrees Celsius, to about 66 degrees Celsius, because of the sensitivity of the laminate to heat, such as when an olefin-based elastomer such as VISTAMAXX™ is utilized, as such polymers may lose some of their elasticity when heated. Even using such low post-bond temperatures and pressures, the present inventors have discovered that a spunbond or meltblown laminate can be formed. Of course, it should be understood that the residence time of the materials may influence the particular bonding parameters employed. In addition, in some embodiments, the nip speed during bonding can range from about 1 foot per minute (fpm) to about 60 fpm, such as from about 10 fpm to about 50 fpm, such as from about 15 fpm to about 40 fpm. Meanwhile, in other embodiments, the nip speed can range from about 100 fpm to about 3000 fpm, such as from about 250 fpm to about 2500 fpm, such as from about 500 fpm to about 2000 fpm.

Generally, as a result of the techniques discussed herein, spunbond or meltblown nonwoven facings containing a polypropylene homopolymer with a polypropylene-based elastomer or a polyethylene homopolymer with a polyethylene-based elastomer. The elastomers can provide the nonwoven facing with the desired level of softness, while at the same time allowing for easier grooving of the nonwoven facing compared to if only polypropylene or polyethylene are utilized, which is a possibility although such facings would be more loosely configured or fuzzy. Because the grooving of such nonwoven facings is easier to accomplish, there is less risk of damaging an underlying elastic film in laminates containing the aforementioned nonwoven facings.

In reference to spunbond nonwoven facings particularly, incorporating an ethylene-based elastomer such as INFUSE™ or a polypropylene-based elastomer such as VERSIFY™ with a polyethylene or a polypropylene creates a softer nonwoven facing that can be more easily grooved than a nonwoven facing containing polyethylene as the only olefinic polymer. Likewise, incorporating a polypropylene-based elastomer such as VISTAMAXX™ with a polypropylene can create a softer nonwoven facing that can be more easily grooved compared to a nonwoven facing containing polypropylene as the only olefinic polymer.

Further, in reference to meltblown nonwoven facings in particular, because meltblown facings generally include polymers having a lower molecular weight than other facings and also are less tacky and not bonded when initially formed, which means that meltblown facings can be grooved more easily. Moreover, polypropylene meltblown facings can be grooved more easily than polyethylene meltblown facings because polypropylene is more brittle than polyethylene, which is softer. In addition, post-bonding of polyethylene-based meltblown facings can be carried out at lower temperatures and pressures because of their lower molecular weights compared to spunbond facings and facings based on polymers other than polyethylene.

However, regardless of whether the facings of the present invention are polyethylene-based, polypropylene-based, spunbond, or meltblown, the film components, facing components, grooving conditions, and bonding conditions can be selected to achieve an elastic nonwoven laminate that has the desired levels of softness and elasticity with reduced fuzziness, while at the same exhibiting enhanced hook engagement and resisting fiber pullout, such as when the elastic nonwoven laminates are used in absorbent article applications utilizing hook or tab fastening means. For instance, when a tab or hook is attached to a laminate of the present invention that has been post bonded with smooth rolls, the elongation at failure (percent elongation) of the tab or hook, which corresponds with hook disengagement, can range from about 50 percent to about 200 percent, such as from about 75 percent to about 190 percent, such as from about 100 percent to about 180 percent. Likewise, when a tab or hook is attached to a laminate of the present invention that has been post-bonded using a wire-weave pattern, the elongation at failure (percent elongation) of the tab or hook can range from about 50 percent to about 150 percent, such as from about 60 percent to about 125 percent, such as from about 70 percent to about 100 percent.

Further, when a tab or hook is attached to a laminate of the present invention that has been post bonded with smooth rolls, the load at failure can range from about 600 grams-force to about 2200 grams-force, such as from about 800 grams-force to about 2100 grams-force, such as from about 1000 grams-force to about 2000 grams-force. Meanwhile, when a tab or hook is attached to a laminate of the present invention that has been post-bonded using a wire-weave pattern, the load at failure can range from about 400 grams-force to about 1200 grams-force, such as from about 500 grams-force to about 1100 grams-force, such as from about 600 grams-force to about 1000 grams-force.

The components of the elastic nonwoven laminates of the present invention can also be selectively controlled to achieve the desired tensile properties. For instance, elastic nonwoven laminates post-bonded with smooth rolls can exhibit a percent elongation of greater than about 200 percent, such as greater than about 400 percent, such as greater than about 800 percent. Further, elastic nonwoven laminates post-bonded using a wire-weave pattern can exhibit a percent elongation of greater than about 200 percent, such as from about 200 percent to about 1000 percent, such as from about 400 percent to about 800 percent. In addition, elastic nonwoven laminates post-bonded using a wire-weave pattern can exhibit a load at failure of greater than about 3000 grams-force, such as greater than about 4000 grams-force, such as greater than about 5000 grams-force. Meanwhile, elastic nonwoven laminates post-bonded using a wire-weave pattern can exhibit a load at failure of from about 1000 grams-force to about 4250 grams-force, such as from about 1500 grams-force to about 4000 grams-force, such as from about 2000 grams-force to about 3750 grams-force.

Further, the laminates of the present invention can exhibit a load loss of less than about 60 percent, such as from about 10 percent to about 60 percent, such as from about 15 percent to about 55 percent, such as from about 30 percent to about 50 percent, which is indicative that even with post-bonding, the laminates of the present invention maintain their elastic properties.

It is also to be understood that the elastic nonwoven laminates of the present disclosure may also include one or more frangible layers located outside the one or more of the facings layers or disposed between the one or more facing layers and the elastic strand. Such frangible layers are described in U.S. patent application Ser. No. 13/720,194, filed on Dec. 19, 2012, which is incorporated herein in its entirety by reference thereto for all purposes. Generally, the frangible layer can also be grooved in the manner described in reference to the nonwoven facings. The frangible layer can be used to add loftiness to the elastic nonwoven laminates of the present invention or to achieve the desired aesthetics depending on the particular application.

The elastic nonwoven laminate of the present invention may be used in a wide variety of applications. For example, the elastic nonwoven laminate may be used in an absorbent article. An “absorbent article” generally refers to any article capable of absorbing water or other fluids. Examples of some absorbent articles include, but are not limited to, personal care absorbent articles, such as diapers, training pants, absorbent underpants, incontinence articles, feminine hygiene products (e.g., sanitary napkins), swim wear, baby wipes, and so forth; medical absorbent articles, such as garments, fenestration materials, underpads, bedpads, bandages, absorbent drapes, and medical wipes; food service wipers; clothing articles; and so forth. Materials and processes suitable for forming such absorbent articles are well known to those skilled in the art. Absorbent articles may include a substantially liquid-impermeable layer (e.g., outer cover), a liquid-permeable layer (e.g., bodyside liner, surge layer, etc.), and an absorbent core. In one particular embodiment, the elastic nonwoven laminate of the present invention may have a wide variety of other uses, such as in providing an elastic waist, leg cuff/gasketing, stretchable ear, side panel, outer cover, or any other component in which elastic properties are desirable.

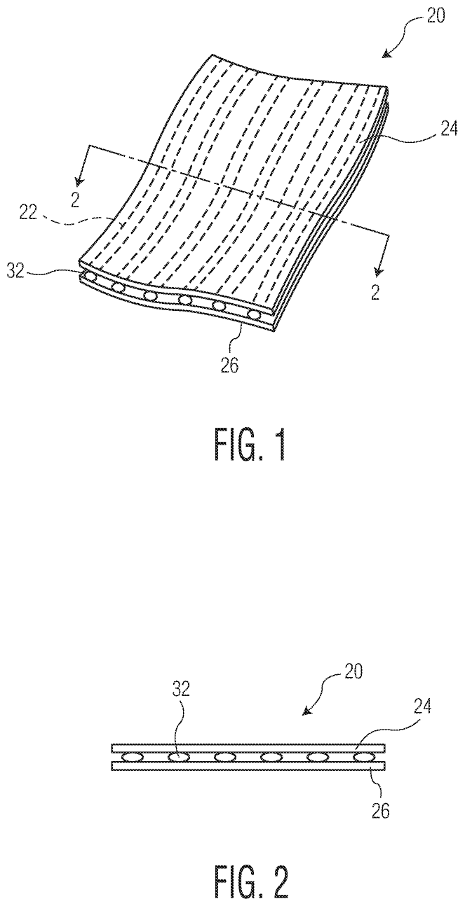

One embodiment of the invention is an elastic stranded laminate 20 of the invention is shown in FIG. 1. The strands 22 may be self-adhered to at least one facing sheet, or between a first facing sheet (or layer) 24 and a second facing sheet (or layer) 26 as shown. A cross-sectional view of the laminate 20 in FIG. 1 is shown in FIG. 2. The strands 22 may be laid out periodically, non-periodically, and in various spacings, groupings, and sizes, according to the effect desired from the elastic strand laminate 20 and the use to which it is used. For example, the strands 22 may be spaced apart to between about 5 and about 15 strands per inch.

The strands 22 are substantially continuous in length. The strands 22 may have a circular cross-section, but may alternatively have other cross-sectional geometries such as elliptical, rectangular as in ribbon-like strands, triangular or multi-lobal. Each strand 22 has a diameter between about 0.1 and about 0.25 inch, with the diameter being the widest cross-sectional dimension of the strand.

The strands 22 made of the elastomeric adhesive composition are capable not only of introducing a degree of elasticity to facing layers 24, 26 but are also capable of providing a construction adhesive function. That is, the strands adhere together the facing layers or other components with which they are in contact. It is also possible that the strands do not constrict upon cooling but, instead, tend to retract to approximately their original dimension after being elongated during use in a product.

Facing layers 24, 26 may be nonwoven webs or polymer films formed using conventional processes, including the spunbond and meltblowing processes described in the DEFINITIONS. For example, the facing layers 24, 26 may each include a spunbonded web having a basis weight of about 0.1-4.0 ounces per square yard (osy), suitably 0.2-2.0 osy, or about 0.4-0.6 osy. The facing layers 24, 26 may include the same or similar materials or different materials.

If the facing layers 24, 26 are to be applied to the strands 22 without first being stretched, the facing layers may or may not be capable of being stretched in at least one direction in order to produce an elasticized area. For example, the facing layers could be necked, or gathered, in order to allow them to be stretched after application of the strands. Various post treatments, such as treatment with grooved rolls, which alter the mechanical properties of the material, are also suitable for use. The laminate 20 suitably has a basis weight between about 20 and about 120 grams per square meter.

Tension within the laminate 20 may be controlled through varying the percentage stretch, or stretch ratio, of the strands 22 prior to adhesion to the facing sheet(s), and/or through the amount of strand add-on or thickness, with greater stretch and greater add-on or thickness each resulting in higher tension. Tension can also be controlled through selection of the elastomeric adhesive composition, and/or by varying strand geometries and/or spacing between strands. For example, holes 32 between the layers 24, 26 through which the composition passes to form strands may vary in diameter. The holes 32 may be from about 0.1 millimeter to about 1 centimeter. The laminate of the invention suitably has tension of at least 100 grams/inch at 100 percent elongation to about 200 grams/inch at 100 percent elongation.

Furthermore, the embossing disclosed herein results in temporary bonding between the two facing layers that are made by meltblown, coform® or spunbond processes. The embossing between the two facing layers permanently deform the fibers, even after the bond between the facing layers are broken. When the laminate is stretched, the “bond points” from the embossing break and strands may be damaged if hit by bond pattern. Accordingly suitable materials are needed to prohibit strand damage. It was unexpectedly found that by using stranded laminates prohibited strand damage.

Materials that were surprisingly found to limit or remove strand damage are disclosed herein. These materials are made with CFSBL (combo of elastic MB and filaments) in addition to materials made with elastic strands only.

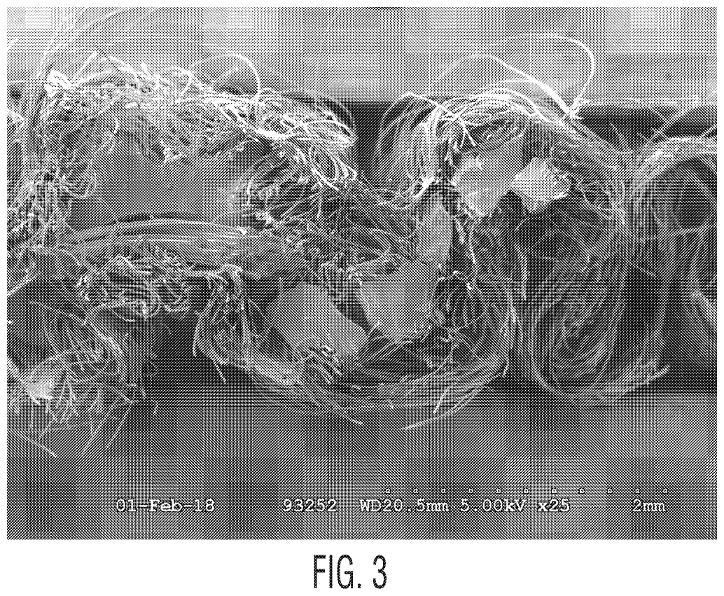

Compared to embossing the laminate, embossing the SB before lamination has a lesser visual effect due to lamination step partially erasing the embossing. The laminate is thicker after embossing versus before. To observe and measure this thickness change, measurements were made via microscopy (for example, using a 3″ plate in a more standard thickness testing) since the thickness change occurs over many small areas. Unlike film based laminates, embossing herein is often done at a lower temperature with using stranded laminates. Samples have been made with VFL and Lycra strands. Microscopy results are available in FIGS. 3 and 4.

In Lycra stranded laminates, the adhesive is on the strands and not in areas between strands. Facing attachment between strands is the result of light thermal bonding.

In VFL stranded laminates, the adhesive may play a part in the attachment of the facing layers since the adhesive is present both on the strands and in areas between strands. In the embossing step, the adhesive can become tacky again resulting in additional adhesion of the materials in the embossing point areas. The adhesive does not attach layers in areas between the embossing points. However, the two facings should have signs of embossing that are aligned with each other.

It is important to note that in either Lycra or VFL stranded laminates the elastic strands and the fibers in the nonwoven facings may be deformed by the embossing step. In that regard, executing the embossing step in line with converting may be disadvantageous versus embossing in line with manufacturing. Alternatively, an elastic film based laminate that includes some strands in distinct regions and the embossing is in the film region—or the embossing is over the entire area may prevent the deformation at the embossing step.

Per FIG. 3, the material disclosed therein has multifilament elastic strands. When the material is stretched, the sample loses little to no elasticity. Per FIG. 4, the material disclosed therein has monofilament elastic strands. When the material is stretched, the sample loses elasticity.

However, in view of FIG. 3 or 4 when viewing away from the embossed areas, the filaments themselves and the adhesive do not seem to change with stretching. They both remain attached to each other and to the nonwoven facings.

### Test Methods

In a SEM in cross-section, the embossed points split apart from stretching. The split occurred in the middle of the thickness and in plane of the material that is parallel to the stretch direction. This is different from using SABBEL and NBL material where the split or damage to the embossed areas could be seen at the surface and occurred in the plane perpendicular to the stretch direction as well.

From transmission light microscopy, it can be seen that the elastic strands are damaged and partially or fully severed in the embossed areas. For the monofilament material in FIG. 4, most of the strands are fully severed in the embossed areas. For the multifilament material in FIG. 3 most of the filaments are not severed in the embossed areas.

In view of FIG. 4, it is concluded that the embossing process does damage the elastic strands. When stretched, the embossed areas split apart. For the material in FIG. 3, however, most or all of the elasticity remains because most or all of the elastic filaments are still intact. For the material in FIG. 4, the embossed areas effectively bridge the area between the severed monofilament strands. Accordingly, the material in FIG. 4 is stretched and the embossed areas is split apart. There is nothing bridging the severed monofilament segments so the material loses its elasticity.

### First Embodiment

In a first embodiment the invention provides for a patterned elastic nonwoven laminate having a machine direction and a cross-machine direction, the elastic nonwoven laminate comprising an unstretched or stretched plurality of strands of an elastomeric construction adhesive composition that is self-adhered to at least a first nonwoven facing layer or a second nonwoven facing layer, wherein the first nonwoven facing layer comprises a first polyolefin and the first nonwoven facing layer is meltblown or spunbond, and wherein the first nonwoven facing layer and the second nonwoven facing layer are simultaneously grooved in the machine direction or cross-machine direction to decouple the first nonwoven facing layer and the second nonwoven facing layer from the elastic strands, wherein grooves in the first nonwoven facing layer correspond with grooves in the second nonwoven facing layer, and wherein an outer surface of each of the first nonwoven facing layer and the second nonwoven facing layer are post-bonded after the first nonwoven facing layer and the second nonwoven facing layer are simultaneously grooved such that from about 1 groove per inch to about 20 grooves per inch are present on the outer surface of each of the first nonwoven facing layer and the second nonwoven facing layer after post-bonding and wherein the outer surface of each of the first nonwoven facing layer and the second nonwoven facing layer are embossed in a pattern, wherein embossed areas of the first nonwoven facing layer and the second nonwoven facing layer are non-fibrous.

The patterned elastic strand laminate according to the preceding embodiment, wherein the elastic nonwoven laminate has a percent elongation of at least about 200 percent in the cross-machine direction.

The patterned elastic strand laminate according to the preceding embodiments, wherein the plurality of strands are spaced apart on at least one facing layer by about 1 to about 10 strands per centimeter.

The patterned elastic strand laminate according to the preceding embodiments, wherein each of the plurality of strands has a diameter between 0.10 and 0.90 centimeter

The patterned elastic strand laminate according to the preceding embodiments, wherein at least one facing sheet comprises a nonwoven web selected from a spunbond web or a meltblown web.

The patterned elastic strand laminate according to the preceding embodiments, wherein the elastomeric adhesive composition further comprises an antioxidant in an amount between about 0.1 percent and about 10.0 percent by weight.

The patterned elastic strand laminate according to the preceding embodiments, wherein the composition has a viscosity of about 10,000 to 50,000 centipoise at between 110 and 130 degrees Celsius.

The patterned elastic strand laminate according to the preceding embodiments, wherein the elastic nonwoven laminate has a basis weight between about 20 and about 120 grams per square meter.

The patterned elastic strand laminate according to the preceding embodiments, wherein the laminate exhibits a stretch-to-stop percent of at least 250 percent.

The patterned elastic strand laminate according to the preceding embodiments, wherein the patterned elastic laminate is used in an absorbent article.

### Second Embodiment

In a second embodiment the invention provides for a process for making a patterned elastic laminate comprising:
stretching a plurality of elastic strands,
positioning the strands between two or more nonwoven facing layers;
attaching the stretched strands to the two or more nonwoven facing layers to form an elastic laminate thereby partially relaxing the elastic laminate;
embossing the relaxed elastic laminate by passing it through the nip of an anvil set of rolls.

The process for making a patterned elastic laminate according to the preceding embodiment, wherein the embossing the relaxed elastic laminate is done by passing it through the nip of a pattern set of rolls.

The process for making a patterned elastic laminate according to preceding second embodiments, wherein the embossing occurs only between strands.

The process for making a patterned elastic laminate according to the preceding second embodiments, wherein the patterned elastic laminate is used in an absorbent article.

### Third Embodiment

In a third embodiment the invention provides for a process for making a patterned elastic laminate, comprising:
stretching a plurality elastic strands;
positioning the strands between two or more nonwoven facing layers and attaching the stretched strands to the facing layers to form an elastic laminate;
keeping the elastic strands in the stretched state; and
embossing the relaxed elastic laminate by passing it through the nip of an anvil set of rolls.

The process for making a patterned elastic laminate according to the third embodiment, wherein embossing the relaxed elastic laminate is done by passing it through the nip of a pattern set of rolls.

The process for making a patterned elastic laminate according to the preceding third embodiments, wherein the embossing occurs only between strands.

The process for making a patterned elastic laminate according to the preceding third embodiments, wherein the patterned elastic laminate is used in an absorbent article.

1. A patterned elastic nonwoven laminate having a machine direction and a cross-machine direction, the elastic nonwoven laminate comprising an unstretched or stretched plurality of strands of an elastomeric construction adhesive composition that is self-adhered to at least a first nonwoven facing layer or a second nonwoven facing layer, wherein the first nonwoven facing layer comprises a first polyolefin and the first nonwoven facing layer is meltblown or spunbond, and wherein the first nonwoven facing layer and the second nonwoven facing layer are simultaneously grooved in the machine direction or cross-machine direction to decouple the first nonwoven facing layer and the second nonwoven facing layer from the elastic strands, wherein grooves in the first nonwoven facing layer correspond with grooves in the second nonwoven facing layer, and wherein an outer surface of each of the first nonwoven facing layer and the second nonwoven facing layer are post-bonded after the first nonwoven facing layer and the second nonwoven facing layer are simultaneously grooved such that from about 1 groove per inch to about 20 grooves per inch are present on the outer surface of each of the first nonwoven facing layer and the second nonwoven facing layer after post-bonding and wherein the outer surface of each of the first nonwoven facing layer and the second nonwoven facing layer are embossed in a pattern, wherein embossed areas of the first nonwoven facing layer and the second nonwoven facing layer are non-fibrous.

2. The elastic strand laminate of claim 1, wherein the elastic nonwoven laminate has a percent elongation of at least 200 percent in the cross-machine direction.

3. The elastic strand laminate of claim 1, wherein the plurality of strands are spaced apart on at least one facing layer by 1 to 10 strands per centimeter.

4. The elastic strand laminate of claim 1, wherein each of the plurality of strands has a diameter between 0.10 and 0.90 centimeter

5. The elastic strand laminate of claim 1, wherein at least one facing sheet comprises a nonwoven web selected from a spunbond web or a meltblown web.

6. The elastic strand laminate of claim 1, wherein the elastomeric adhesive composition further comprises an antioxidant in an amount between 0.1 percent and 10.0 percent by weight.

7. The elastic strand laminate of claim 1, wherein the composition has a viscosity of 10,000 to 50,000 centipoise at between 110 and 130 degrees Celsius.

8. The elastic strand laminate of claim 1, wherein the elastic nonwoven laminate has a basis weight between 20 and 120 grams per square meter.

9. The elastic strand laminate of claim 1, wherein the laminate exhibits a stretch-to-stop percent of at least 250 percent.

10. The elastic laminate according to claim 1, wherein the patterned elastic laminate is used in an absorbent article.

11. A process for making a patterned elastic laminate comprising:
stretching a plurality of elastic strands,
positioning the strands between two or more nonwoven facing layers;
attaching the stretched strands to the two or more nonwoven facing layers to form an elastic laminate thereby partially relaxing the elastic laminate;
embossing the relaxed elastic laminate by passing it through the nip of an anvil set of rolls, wherein the embossing occurs only between strands.

12. The process for making a patterned elastic laminate according to claim 10, wherein the embossing the relaxed elastic laminate is done by passing it through the nip of a pattern set of rolls.

13. (canceled)

13. The process for making a patterned elastic laminate according to claim 10, wherein the patterned elastic laminate is used in an absorbent article.

14. A process for making a patterned elastic laminate, comprising:
stretching a plurality elastic strands;
positioning the strands between two or more nonwoven facing layers and attaching the stretched strands to the facing layers to form an elastic laminate;
keeping the elastic strands in the stretched state; and
relaxing and embossing the elastic laminate by passing it through the nip of an anvil set of rolls, wherein the embossing occurs only between strands.

15. The process for making a patterned elastic laminate according to claim 14, wherein embossing the relaxed elastic laminate is done by passing it through the nip of a pattern set of rolls.

17. (canceled)

16. The process for making a patterned elastic laminate according to claim 14, wherein the patterned elastic laminate is used in an absorbent article.
